(19) United States
(12) Patent Application Publication
(10) Pub. No.: US 2010/0272675 A1
(43) Pub. Date: Oct. 28, 2010

(54) METHOD OF ADMINISTERING CONJUGATES

(75) Inventors: Christopher Paul Leamon, West Lafayette, IN (US); P. Ronald Ellis, West Lafayette, IN (US)

Correspondence Address: BARNES & THORNBURG LLP, 11 SOUTH MERIDIAN, INDIANAPOLIS, IN, 46204, US

(73) Assignee: ENDOCYTE, INC., West Lafayette, IN (US)

(21) Appl. No.: 12/743,191

(22) PCT Filed: Nov. 14, 2008
(86) PCT No.: PCT/US08/83580

§ 371 Date: May 14, 2010

Related U.S. Application Data
(60) Provisional application No. 61/003,212, filed on Nov. 15, 2007.
(60) Provisional application No. 60/990,815, filed on Nov. 28, 2007.
(60) Provisional application No. 61/043,833, filed on Apr. 10, 2008.
(60) Provisional application No. 60/988,621, filed on Nov. 16, 2007.

(51) Int. Cl. A61K 38/20; A61K 47/00; A61K 38/21

(52) U.S. Cl. 424/85.2; 424/278.1; 424/85.5; 424/85.7

(57) ABSTRACT

The invention relates to a method of treating a host animal to eliminate pathogenic cells. The method comprises the steps of administering to the host animal a hapten-carrier conjugate, administering to the host animal a TH-I biasing adjuvant, and administering to said host animal a ligand conjugated to a hapten herein the ligand-hapten conjugate is administered during the first cycle of therapy with the hapten-carrier conjugate. The invention also relates to the same method wherein the ratio of the hapten-carrier conjugate to the TH-I biasing adjuvant on a weight to weight basis ranges from about 1:10 to about 1:1.

### CROSS REFERENCE TO RELATED APPLICATIONS

This application claims the benefit under 35 U.S.C. §119(e) of U.S. Provisional Application Ser. Nos. 61/003,212, filed on Nov. 15, 2007, 60/988,621, filed on Nov. 16, 2007, 60/990,815, filed on Nov. 28, 2007, and 61/043,833, filed on Apr. 10, 2008 each application incorporated herein by reference.

### FIELD OF THE INVENTION

The invention relates to methods of administering ligand conjugates for use in treating disease states caused by pathogenic cells. More particularly, targeted ligand-immunogen conjugates are administered to a diseased host to treat diseases such as cancer, inflammation, and other diseases caused by activated immune cells.

### BACKGROUND AND SUMMARY OF THE INVENTION

The mammalian immune system provides a means for the recognition and elimination of tumor cells, and other pathogenic cells. While the immune system normally provides a strong line of defense, there are still many instances where cancer cells, and other pathogenic cells evade the host immune response and persist with concomitant host pathogenicity. Chemotherapeutic agents and radiation therapies have been developed to eliminate replicating cancer cells. However, most, if not all, of the currently available chemotherapeutic agents and radiation therapy regimens have adverse side effects because they work not only to destroy cancer cells, but they also affect normal host cells, such as cells of the hematopoietic system. Moreover, resistance to chemotherapeutic agents can develop. The capacity of cancer cells to develop resistance to therapeutic agents, and the adverse side effects of the currently available anticancer drugs, highlight the need for the development of new targeted therapies with specificity and reduced host toxicity.

The methods described herein are directed to eliminating pathogenic cell populations in a host by increasing host immune system recognition of and response to such cell populations. Effectively, the antigenicity of the pathogenic cells is increased to enhance the endogenous immune response-mediated elimination of the pathogenic cells. The method comprises administration of a ligand-immunogen conjugate wherein the ligand is capable of specific binding to a population of pathogenic cells in vivo that uniquely expresses, preferentially expresses, or overexpresses a ligand binding moiety, and the ligand conjugated immunogen is capable of eliciting antibody production or is capable of being recognized by endogenous or co-administered exogenous antibodies in the host animal. The immune system-mediated elimination of the pathogenic cells is directed by the binding of the immunogen conjugated ligand to a receptor, a transporter, or other surface-presented protein uniquely expressed, overexpressed, or preferentially expressed by the pathogenic cell. A surface-presented protein uniquely expressed, overexpressed, or preferentially expressed by the pathogenic cell is a receptor not present or present at low amounts on non-pathogenic cells providing a means for selective elimination of the pathogenic cells. At least one additional therapeutic factor, for example, an immune system stimulant, a cell killing agent, a tumor penetration enhancer, a chemotherapeutic agent, or a cytotoxic immune cell may be co-administered to the host animal to enhance therapeutic efficiency.

In one embodiment, a method of treating a host animal to eliminate pathogenic cells is provided. The method comprises the steps of administering to the host animal a hapten-carrier conjugate, administering to the host animal a T_{H}-1 biasing adjuvant wherein the ratio of the hapten-carrier conjugate to the T_{H}-1 biasing adjuvant on a weight to weight basis ranges from about 1:10 to about 1:1, and administering to the host animal a ligand conjugated to a hapten wherein the ligand-hapten conjugate is administered during the first week of administration of the hapten-carrier conjugate, or at a later time wherein the later time is before the first cycle of therapy with the hapten-carrier conjugate is complete. In additional embodiments, the pathogenic cells are cancer cells, the pathogenic cells are activated immune cells, or the activated immune cells are macrophages or monocytes. In another embodiment, the ligand-hapten conjugate is administered during the first second, third, or fourth week of administration of the hapten-carrier conjugate.

In yet other embodiments, the ligand is a vitamin receptor binding ligand, the ligand is selected from the group consisting of folic acid and other folate receptor-binding ligands, the ligand is a folic acid analog having a glutamyl moiety covalently linked to the hapten only via the glutamyl γ-carboxyl moiety of the ligand, the ligand is a folic acid analog having a glutamyl moiety covalently linked to the hapten only via the glutamyl α-carboxyl moiety of the ligand, or the ligand is a small organic molecule capable of binding to a receptor and wherein said receptor is preferentially expressed, uniquely expressed or overexpressed on the surface of said population of pathogenic cells. In other aspects, the hapten is an organic molecule having a molecular weight less than 20,000 daltons, and/or the organic molecule is selected from the group consisting of fluorescein, a nitrophenyl, and a polynitrophenyl.

In other illustrative aspects, the method further comprises the step of administering an immune stimulant to the host animal, the immune stimulant is a cytokine, the cytokine comprises IL-2, IL-12, IL-15, or combinations thereof, or the cytokine comprises IL-2, IL-12, IL-15, or combinations thereof, in combination with IFN-γ or IFN-α. In other embodiments, the ligand-hapten conjugate composition is administered in multiple injections, the administration of the hapten-carrier conjugate comprises a vaccination, and/or the ratio of the hapten-carrier conjugate to the T_{H}-1 biasing adjuvant on a weight to weight basis ranges from about 1:8 to about 1:1, about 1:6 to about 1:1, about 1:4 to about 1:1, about 1:3 to about 1:1, or is about 1:3 or about 1:2.5.

In another illustrative embodiment, the adjuvant is a quillajasaponin adjuvant, the adjuvant is a modified saponin adjuvant, the carrier is keyhole limpet hemocyanin, or the hapten-carrier conjugate has the formula:

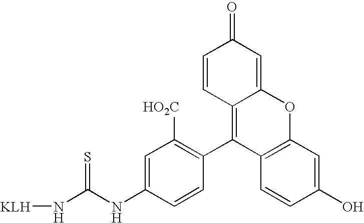

wherein KLH is keyhole limpet hemocyanin, and the ligand-hapten conjugate has the formula:

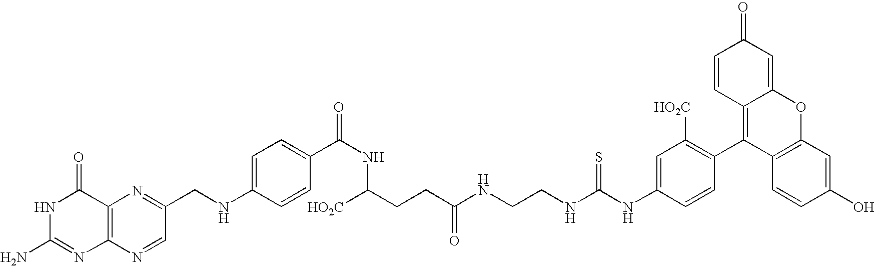

or pharmaceutically acceptable salts thereof.

In any of the above-described embodiments, a method of treating a host animal to eliminate pathogenic cells is provided wherein the method comprises the steps of administering to the host animal a hapten-carrier conjugate, administering to the host animal a T_{H}-1 biasing adjuvant, and administering to the host animal a ligand conjugated to a hapten wherein the ligand-hapten conjugate is administered during the first cycle of therapy with the hapten-carrier conjugate.

### BRIEF DESCRIPTION OF THE DRAWINGS

FIG. 1 shows the results of an assay where rectal temperatures in mice injected with Bis-EDA-FITC along with folate-FITC were measured with early or late dosing of folate-FITC. The mice were preimmunized with 1 μg doses of KLH-FITC.

FIG. 2 shows rectal temperatures in mice injected with Bis-EDA-FITC along with folate-FITC with early or late dosing of folate-FITC. The mice were preimmunized with 35 μg doses of KLH-FITC.

FIG. 3 shows the effect of folate-targeted immunotherapy on the survival of mice with breast tumor implants using early or late dosing of folate-FITC. The mice were preimmunized with 35 μg doses of KLH-FITC.

FIG. 4 shows an exemplary structure of folate-FITC.

FIG. 5 shows an exemplary structure of KLH-FITC.

FIG. 6 shows a KLH-FITC versus folate-FITC dosing protocol.

FIG. 7 shows an exemplary dosing schematic. Panel A: a single dose of EC17 was intravenously administered on Day 23. Panel B: mice were de-sensitized with multiple subcutaneous doses of EC17 on Days 8-12, 15-19, and 22.

FIG. 8 shows anti-FITC IgE antibody production in immunized mice.

FIG. 9 shows an anaphylaxis assay in immunized guinea pigs.

### DETAILED DESCRIPTION OF THE INVENTION

Methods are provided for the therapeutic treatment of a host with cancer or a disease state caused by activated immune cells, such as macrophages or monocytes. The methods result in enhancement of the immune response-mediated elimination of pathogenic cells by labeling the pathogenic cells antigenic resulting in their recognition and elimination by the host immune system. The method employs a ligand-immunogen conjugate capable of high affinity binding to cancer cells or other pathogenic cells, such as activated immune cells. The ligand-immunogen conjugate decorates the pathogenic cells so that they appear antigenic and are eliminated by the host's own immune system or by, for example, co-administered antibodies. The method may also utilize combination therapy by employing the ligand-immunogen conjugate and an additional therapeutic factor capable of stimulating an endogenous immune response (e.g., an immune stimulant such as a cytokine).

The method described herein is utilized to enhance an endogenous immune response-mediated elimination of a population of pathogenic cells in a host animal harboring the population of pathogenic cells. The invention is applicable to populations of pathogenic cells that cause a variety of pathologies such as cancer and inflammation. In various aspects, the population of pathogenic cells may be a cancer cell population that is tumorigenic, including benign tumors and malignant tumors, or it can be non-tumorigenic. In other embodiments, the cancer cell population may arise spontaneously or by such processes as mutations present in the germline of the host animal or somatic mutations, or it may be chemically-, virally-, or radiation-induced. In other illustrative embodiments, the methods can be utilized to treat such cancers as carcinomas, sarcomas, lymphomas, Hodgekin's disease, melanomas, mesotheliomas, Burkitt's lymphoma, nasopharyngeal carcinomas, leukemias, and myelomas. In various other embodiments, the cancer cell population can include, but is not limited to, oral, thyroid, endocrine, skin, gastric, esophageal, laryngeal, pancreatic, colon, bladder, bone, ovarian, cervical, uterine, breast, testicular, prostate, rectal, kidney, liver, and lung cancers.

The methods described herein can be used for both human clinical medicine and veterinary applications. In various illustrative aspects, the host animals harboring the population of pathogenic cells and treated with ligand-immunogen conjugates may be humans (e.g., a human patient) or, in the case of veterinary applications, may be laboratory, agricultural, domestic, or wild animals.

In various illustrative embodiments, the ligand-immunogen conjugate may be administered to the host animal parenterally, e.g., intradermally, subcutaneously, intramuscularly, intraperitoneally, or intravenously. In other embodiments, the conjugate may be administered to the host animal by other medically useful processes, and any effective dose and suitable therapeutic dosage form, including prolonged release dosage forms, can be used. Illustratively, the method described herein may be used in combination with surgical removal of a tumor, radiation therapy, chemotherapy, or biological therapies such as other immunotherapies including, but not limited to, monoclonal antibody therapy, treatment with immunomodulatory agents, adoptive transfer of immune effector cells, treatment with hematopoietic growth factors, cytokines and vaccination.

In accordance with the methods described herein, the ligand-immunogen conjugates may be selected from a wide variety of ligands and immunogens. The ligands can be capable of specific binding to the pathogenic cells in the host animal due to preferential expression of a receptor for the ligand, accessible for ligand binding, on the pathogenic cells. In various exemplary embodiments, acceptable ligands include folic acid, analogs of folic acid and other folate receptor-binding molecules, other vitamins, peptide ligands identified from library screens, tumor-specific peptides, tumor-specific aptamers, tumor-specific carbohydrates, tumor-specific monoclonal or polyclonal antibodies, Fab or scFv (i.e., a single chain variable region) fragments of antibodies or other proteins specifically expressed or uniquely accessible on metastatic cancer cells, small organic molecules derived from combinatorial libraries, growth factors, such as EGF, FGF, insulin, and insulin-like growth factors, and homologous polypeptides, somatostatin and its analogs, transferrin, lipoprotein complexes, bile salts, selectins, steroid hormones, Arg-Gly-Asp containing peptides, retinoids, various Galectins, γ-opioid receptor ligands, cholecystokinin A receptor ligands, ligands specific for angiotensin AT1 or AT2 receptors, peroxisome proliferator-activated receptor γ ligands, and other molecules that bind specifically to a receptor preferentially expressed on the surface of tumor cells or activated immune cells, or fragments of any of these molecules. As used herein, “folate receptor binding ligands” includes any ligand capable of high affinity binding to the folate receptor, including folate receptor-binding analogs and derivatives.

In various embodiments, a folate receptor binding ligand can be folic acid, a folic acid analog, or another folate receptor-binding molecule. Analogs of folate that can be used include folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refers to the art recognized analogs having a carbon atom substituted for one or two nitrogen atoms in the naturally occurring folic acid structure. For example, the deaza analogs include the 1-deaza, 3-deaza, 5-deaza, 8-deaza, and 10-deaza analogs. The dideaza analogs include, for example, 1,5 dideaza, 5,10-dideaza, 8,10-dideaza, and 5,8-dideaza analogs. The foregoing folic acid analogs are conventionally termed “folates,” reflecting their capacity to bind to folate receptors. Other folate receptor-binding analogs include aminopterin, amethopterin (methotrexate), N¹⁰-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate). Any other folate receptor binding analog or derivative such as those described in U.S. Pat. Nos. 2,816,110, 5,140,104, 5,552,545, or 6,335,434, incorporated herein by reference, can also be used. Any folate analog or derivative well-known in the art, such as those described in Westerhof, et al., Mol. Pharm. 48: 459-471 (1995), incorporated herein by reference can be used.

Additional illustrative analogs of folic acid that bind to folic acid receptors (i.e., folate receptor binding ligands) are described in U.S. Patent Application Publication Serial Nos. 2005/0227985 and 2004/0242582, the disclosures of which are incorporated herein by reference. Illustratively, such folate analogs have the general formula, where the (*) represents the point of attachment of additional bivalent linker radicals:

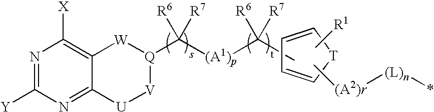

wherein X and Y are each—independently selected from the group consisting of halo, R², OR², SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;

A¹ and A² are each independently selected from the group consisting of oxygen, sulfur, —C(Z)-, —C(Z)O—, —OC(Z)-, —N(R^{4b})—, —C(Z)N(R^{4b})—, —N(R^{4b})C(Z)-, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C ≡CH)—, —N(CH₂C≡CH)—, C₁-C₁₂ alkylene, and C₁-C₁₂ alkyeneoxy, where Z is oxygen or sulfur;

R¹ is selected from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

L is a bivalent linker as described herein; and

n, p, r, s and t are each independently either 0 or 1.

In one aspect of such folate receptor binding analogs of folate, when s is 1, t is 0, and when s is 0, t is 1. In another aspect of such folate analogs, both n and r are 1, and linker L^{a} is a naturally occurring amino acid covalently linked to A² at its alpha-amino group through an amide bond. Illustrative amino acids include aspartic acid, glutamic acid, and the like.

The foregoing folic acid analogs and/or derivatives are conventionally termed “folates,” reflecting their ability to bind with folate-receptors, and such ligands when conjugated with exogenous molecules are effective to enhance transmembrane transport, such as via folate-mediated endocytosis as described herein. Accordingly, as used herein, it is to be understood that the term “folate” refers both individually to folic acid used in forming a conjugate, or alternatively to a folate analog or derivative thereof that is capable of binding to folate or folic acid receptors (i.e., folate receptor binding ligands).

In another embodiment, other vitamins can be used as the ligand. For example, the vitamins that can be used in accordance with the methods described herein include niacin, pantothenic acid, folic acid, riboflavin, thiamine, biotin, vitamin B₁₂, vitamins A, D, E and K, other related vitamin molecules, analogs and derivatives thereof, and combinations thereof (see U.S. Pat. Nos. 5,108,921, 5,416,016, and 5,635,382 incorporated herein by reference).

In one illustrative aspect, the binding site for the ligand may include receptors for any molecule capable of specifically binding to a receptor wherein the receptor or other protein is preferentially expressed on the population of pathogenic cells, including, for example, cancer cells or activated immune cells. In various embodiments, the binding sites can be receptors for growth factors, vitamins, peptides, including opioid peptides, hormones, antibodies, carbohydrates, or small organic molecules, or the binding sites may be tumor-specific antigens. In one embodiment, a combination of ligand-immunogen conjugates can be used to maximize targeting of the pathogenic cells for elimination by the host's immune response or by co-administered antibodies.

In various embodiments of the methods described herein a preexisting immunity or an immunity that constitutes part of the innate immune system can be employed. In another embodiment, antibodies directed against the immunogen may be administered to the host animal to establish a passive immunity. In illustrative aspects, suitable immunogens for use in the invention include antigens or antigenic peptides against which a preexisting immunity has developed via normally scheduled vaccinations or prior natural exposure to such agents as poliovirus, tetanus, typhus, rubella, measles, mumps, pertussis, tuberculosis, and influenza antigens, and α-galactosyl groups. In such cases, the ligand-immunogen conjugates will be used to redirect a previously acquired humoral or cellular immunity to the pathogenic cells in the host animal for elimination of the foreign cells or pathogenic organisms. In other embodiments, the immunogen can be an antigen or antigenic peptide to which the host animal has developed a novel immunity through immunization against an unnatural antigen or hapten (e.g., fluorescein isothiocyanate, dinitrophenyl, or trinitrophenyl) and antigens against which an innate immunity exists (e.g., super antigens and muramyl dipeptide) or, for example, a small organic molecule having a molecular weight less than 20,000 daltons. As used herein, an “immunogen” is a compound that is not an antibody, and an immunogen is a compound that a physician administers in order to elicit an IgG or an IgM antibody response to cause a therapeutic response in a therapeutic method.

In various illustrative aspects, the ligands and immunogens of the invention may be conjugated by utilizing any art-recognized method of forming a conjugate, including covalent, ionic, or hydrogen bonding of the ligand to the immunogen, either directly or indirectly via a linking group such as a divalent linker. For example, the conjugate is typically formed by covalent bonding of the ligand to the immunogen through the formation of amide, ester or imino bonds between acid, aldehyde, hydroxy, amino, or hydrazo groups on the respective components of the complex. Methods of linking ligands to immunogens are described in PCT Publication No. WO 2006/012527, incorporated herein by reference.

In addition, in various embodiments structural modifications of the linker portion of the conjugates are made. For example, a number of amino acid substitutions may be made to the linker portion of the conjugate, including but not limited to naturally occurring amino acids, as well as those available from conventional synthetic methods. In one aspect, beta, gamma, and longer chain amino acids may be used in place of one or more alpha amino acids. In another aspect, the stereochemistry of the chiral centers found in such molecules may be selected to form various mixture of optical purity of the entire molecule, or only of a subset of the chiral centers present. In another aspect, the length of the peptide chain included in the linker may be shortened or lengthened, either by changing the number of amino acids included therein, or by including more or fewer beta, gamma, or longer chain amino acids. In another aspect, the selection of amino acid side chains in the peptide portion may be made to increase or decrease the relative hydrophilicity of the linker portion specifically, or of the overall molecule generally.

Similarly, the length and shape of other chemical fragments of the linkers described herein may be modified. In one aspect, the linker includes an alkylene chain. The alkylene chain may vary in length, or may include branched groups, or may include a cyclic portion, which may be in line or spiro relative to the alkylene chain.

In one embodiment, the ligand is folic acid, an analog of folic acid, or any other folate-receptor binding molecule, and the folate ligand is conjugated to the immunogen by a procedure that utilizes trifluoroacetic anhydride to prepare γ-esters of folic acid via a pteroyl azide intermediate resulting in the synthesis of a folate ligand, conjugated to the immunogen only through the γ-carboxy group of the glutamic acid groups of folate wherein the γ-conjugate binds to the folate receptor with high affinity, avoiding the formation of mixtures of a γ-conjugate and an α-conjugate.

In another embodiment, α-conjugates can be prepared from intermediates wherein the γ-carboxy group is selectively blocked, the α-conjugate is formed and the γ-carboxy group is subsequently deblocked using art-recognized organic synthesis protocols and procedures.

In the methods described herein, the ligand-immunogen conjugates enhance an endogenous immune response-mediated elimination of the pathogenic cells. For example, the endogenous immune response may include a humoral response, a cell-mediated immune response, and any other immune response endogenous to the host animal, including complement-mediated cell lysis, antibody-dependent cell-mediated cytoxicity (ADCC), antibody opsonization leading to phagocytosis, clustering of receptors upon antibody binding resulting in signaling of apoptosis, antiproliferation, or differentiation, and direct immune cell recognition of the delivered antigen/hapten. In various aspects, the endogenous immune response may include the participation of such immune cell types as B cells, T cells, including helper and cytotoxic T cells, macrophages, natural killer cells, neutrophils, LAK cells and the like.

In one embodiment, the humoral response may be a response induced by such processes as normally scheduled vaccination, or active immunization with a natural antigen or an unnatural antigen or hapten (e.g., fluorescein isothiocyanate, a nitrophenyl, or a polynitrophenyl (e.g., dinitrophenyl or trinitrophenyl)) with the unnatural antigen or hapten inducing a novel immunity. For example, active immunization can involve multiple injections of the natural antigen, unnatural antigen or hapten scheduled outside of a normal vaccination regimen to induce the novel immunity. In accordance with the methods described herein, the natural antigen, unnatural antigen, or hapten can be administered in combination with an adjuvant (in the same or different solutions), such as a quillajasaponin adjuvant (e.g., GPI-0100) or any other T_{H}-1 biasing adjuvant.

In one embodiment, the host is preimmunized with a hapten-carrier (e.g., KLH or BSA) conjugate and a T_{H}1-biasing adjuvant to elicit a preexisting immunity to the hapten. The ligand-hapten conjugate is then administered to the host resulting in an humoral or cell-mediated immune response, or both, directed against the ligand-hapten conjugate bound to the targeted pathogenic cells. In one aspect, the host is preimmunized with the hapten-carrier conjugate and the T_{H}1-biasing adjuvant in combination, in the same or different solutions. In this embodiment, the T_{H}1-biasing adjuvant enhances the immune response to the hapten upon subsequent administration of the ligand-hapten conjugate.

Exemplary carriers that can be used include keyhole limpet hemocyanin (KLH), haliotis tuberculata hemocyanin (HtH), inactivated diptheria toxin, inactivated tetanus toxoid, purified protein derivative (PPD) of Mycobacterium tuberculosis, bovine serum albumin (BSA), ovalbumin (OVA), g-globulins, thyroglobulin, peptide antigens, and synthetic carriers, such as poly-L-lysine, dendrimer, and liposomes.

In embodiments where a hapten is used, the hapten is typically conjugated to a carrier to form a hapten-carrier conjugate. The hapten and carrier can be conjugated using any of the methods described above. For example, the carrier (e.g., KLH or BSA) can be conjugated to the hapten by using any art-recognized method of forming a complex including covalent, ionic, or hydrogen bonding of the carrier to the hapten, either directly or indirectly via a linking group such as a divalent linker. The hapten-carrier conjugate is typically formed by covalent bonding through the formation of amide, ester or imino bonds between acid, aldehyde, hydroxy, amino, or hydrazo groups on the respective components of the conjugates. In embodiments where a linker is used, the linker typically comprises about 1 to about 30 carbon atoms, more typically about 2 to about 20 carbon atoms. Lower molecular weight linkers (i.e., those having an approximate molecular weight of about 20 to about 500) are typically employed. In another embodiment, the linker can comprise an indirect means for associating the carrier with the hapten, such as by connection through intermediary linkers, spacer arms, or bridging molecules.

In the embodiment where a hapten-carrier conjugate (see, for example, FIG. 5) is used, the ratio of the hapten-carrier conjugate to the T_{H}-1 biasing adjuvant on a weight to weight basis can range from about 1:10 to about 1:1, about 1:8 to about 1:1, about 1:6 to about 1:1, about 1:4 to about 1:1, about 1:3 to about 1:1, or can be about 1:3 or about 1:2.5. In other illustrative aspects where a hapten-carrier conjugate is used, the molar ratio of the hapten-carrier conjugate to the T_{H}-1 biasing adjuvant can range from about 1.0×10⁻³ to about 6×10⁻⁵.

In one embodiment, adjuvants that bias the immune response towards a T_{H}1 response can be used. An adjuvant-induced T_{H}1-biased immunity can be measured in mice through immunoglobulin isotype distribution analysis. Adjuvants that bias the immune response towards a T_{H}1 response are adjuvants that preferentially increase IgG2a antibody levels in mice relative to IgG 1 antibody levels. An antigen-specific IgG2a/IgG1 ratio of ≧1 can be indicative of a T_{H}1-like antibody subclass pattern. However, in accordance with the invention, any adjuvant that increases the production of antigen-specific antibodies, and, at the same time, increases the relative IgG2a/IgG1 ratio to about ≧0.3 in mice drives the immune response towards a T_{H}1-biased immune response. In various aspects, such adjuvants can include saponin adjuvants (e.g., the quillajasaponins, including lipid-modified quillajasaponin adjuvants), CpG, 3-deacylated monophosphoryl lipid A (MPL), Bovine Calmette-Guerin (BCG), double stem-loop immunomodulating oligodeoxyribonucleotides (d-SLIM), heat-killed Brucella abortus (HKBA), heat-killed Mycobacterium vaccae (SRL172), inactivated vaccinia virus, cyclophosphamide, prolactin, thalidomide, actimid, revimid, and the like. Saponin adjuvants and methods of their preparation and use are described in detail in U.S. Pat. Nos. 5,057,540, 5,273,965, 5,443,829, 5,508,310, 5,583,112, 5,650,398, 5,977,081, 6,080,725, 6,231,859, and 6,262,029 incorporated herein by reference.

In another embodiment, the humoral response may result from an innate immunity where the host animal has a natural preexisting immunity, such as an immunity to α-galactosyl groups. In another illustrative aspect, a passive immunity may be established by administering antibodies to the host animal such as natural antibodies collected from serum or monoclonal antibodies that may or may not be genetically engineered antibodies, including humanized antibodies. The utilization of a particular amount of an antibody reagent to develop a passive immunity, and the use of a ligand-immunogen conjugate wherein the passively administered antibodies are directed to the immunogen, would provide the advantage of a standard set of reagents to be used in cases where a patient's preexisting antibody titer to other potential antigens is not therapeutically useful. In one embodiment, the passively administered antibodies may be “co-administered” with the ligand-immunogen conjugate and co-administration is defined as administration of antibodies at a time prior to, at the same time as, or at a time following administration of the ligand-immunogen conjugate.

The preexisting antibodies, induced antibodies, or passively administered antibodies are redirected to the tumor cells or other pathogenic cells by preferential binding of the ligand-immunogen conjugates to these invading cells. Illustratively, the pathogenic cells can be eliminated by complement-mediated lysis, ADCC, antibody-dependent phagocytosis, or antibody clustering of receptors. The cytotoxic process may also involve other types of immune responses, such as cell-mediated immunity, as well as secondary responses that arise when the attracted antigen-presenting cells phagocytose the unwanted cells and present natural tumor antigens to the immune system for elimination of the cells or organisms bearing the antigens. As used herein, the terms “eliminated” and “eliminating” in reference to the disease state, mean reducing the symptoms or eliminating the symptoms of the disease state or preventing the progression or the reoccurrence of disease. As used herein, the terms “elimination” and “deactivation” of the immune cell population that expresses the ligand receptor mean that this cell population is killed or is completely or partially inactivated which reduces the immune cell-mediated pathogenesis characteristic of the disease state being treated.

In one illustrative aspect, at least one additional composition comprising a therapeutic factor may be administered to the host in combination with the above-detailed methodology, to enhance the endogenous immune response-mediated elimination of the pathogenic cells, or more than one additional therapeutic factor may be administered. The therapeutic factor may be selected from a compound capable of stimulating an endogenous immune response, a chemotherapeutic agent, or other therapeutic factor capable of complementing the efficacy of the administered ligand-immunogen complex. In this embodiment, the additional therapeutic factor can be capable of stimulating an endogenous immune response such as cytokines or immune cell growth factors such as interleukins 1-18, stem cell factor, basic FGF, EGF, G-CSF, GM-CSF, FLK-2 ligand, HILDA, MIP-1α, TGF-β, TGF-α, M-CSF, IFN-γ; IFN-α, IFN-β, soluble CD23, LIF, and combinations thereof.

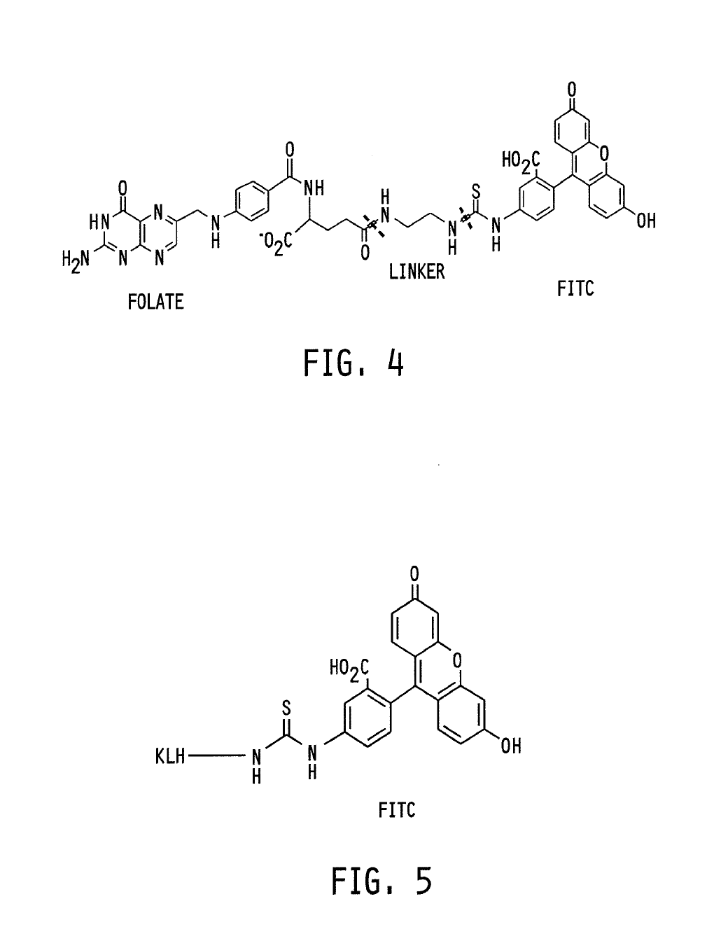

In one embodiment, for example, therapeutically effective amounts of IL-2, for example, in amounts ranging from about 5000 IU/dose/day to about 500,000 IU/dose/day in a multiple dose daily regimen, and IFN-α, for example, in amounts ranging from about 7500 IU/dose/day to about 150,000 IU/dose/day in a multiple dose daily regimen, are used along with folate-FITC (see FIG. 4) to eliminate pathogenic cells in a host animal harboring such a population of cells. In another aspect, therapeutically effective amounts of IL-2 can be used, for example, in amounts ranging from about 0.1 MIU/m²/dose/day to about 60 MIU/m²/dose/day in a multiple dose daily regimen, and IFN-α, for example, in amounts ranging from about 0.1 MIU/m²/dose/day to about 10 MIU/m²/dose/day in a multiple dose daily regimen, can be used (MIU=million international units; m²=approximate body surface area of an average human). In another embodiment, IL-2 and IFN-α are used in therapeutically effective amounts (e.g., 7 MIU and 3 MIU, respectively), and in yet another embodiment IL-15 and IFN-γ are used in therapeutically effective amounts. In an alternate embodiment, IL-2, IFN-γ, or IFN-α, and GM-CSF are used in combination. In other embodiments, any other effective combination of cytokines including combinations of other interleukins and interferons and colony stimulating factors can be used.

In other illustrative embodiments, chemotherapeutic agents, which are cytotoxic themselves and can work to enhance tumor permeability, or reduce allergenicity, suitable for use in the method described herein include adrenocorticoids, alkylating agents, antiandrogens, antiestrogens, corticosteroids, diphenhydramine, androgens, estrogens, antimetabolites such as cytosine arabinoside, purine analogs, pyrimidine analogs, and methotrexate, busulfan, carboplatin, chlorambucil, cisplatin and other platinum compounds, tamoxiphen, taxol, cyclophosphamide, plant alkaloids, prednisone, hydroxyurea, teniposide, antibiotics such as mitomycin C and bleomycin, nitrogen mustards, nitrosureas, vincristine, vinblastine, inflammatory and proinflammatory agents, antihistamines, and any other art-recognized chemotherapeutic agent or agent that reduces allergenicity.

Illustratively, the elimination of the pathogenic cells can comprise a reduction or elimination of tumor mass or of pathogenic immune cells resulting in a therapeutic response. In the case of a tumor, the elimination may be an elimination of cells of the primary tumor or of cells that have metastasized or are in the process of dissociating from the primary tumor. In one embodiment, a prophylactic treatment to prevent return of a tumor after its removal by any therapeutic approach including surgical removal of the tumor, radiation therapy, chemotherapy, or biological therapy is also provided. The prophylactic treatment may be an initial treatment with the ligand-immunogen conjugate, such as treatment in a multiple dose daily regimen, and/or may be an additional treatment or series of treatments after an interval of days or months following the initial treatments(s).

In various embodiments, the unitary daily dosage of the ligand-immunogen conjugate can vary significantly depending on the host condition, the disease state being treated, the molecular weight of the conjugate, its route of administration and tissue distribution, and the possibility of co-usage of other therapeutic treatments such as radiation therapy. The effective amount to be administered to a patient is based on body surface area, patient weight, and physician assessment of patient condition. In various exemplary embodiments, an effective dose can range from about 1 ng/kg to about 1 mg/kg, from about 1 μg/kg to about 500 μg/kg, or from about 100 μg/kg to about 400 μg/kg (e.g., about 300 μg/kg).

Illustratively, the dosages of the adjuvant and the hapten-carrier conjugate can vary depending on the host condition, the disease state being treated, the molecular weight of the conjugate, route of administration and tissue distribution, and the possibility of co-usage of other therapeutic treatments such as radiation therapy. The effective amounts to be administered to a patient are based on body surface area, patient weight, and physician assessment of patient condition. In one illustrative aspect, effective doses of the adjuvant can range from about 0.01 μg to about 100 mg per dose, or from about 100 μg to about 50 mg per dose, or from about 500 μg to about 10 mg per dose or from about 1 mg to 10 mg per dose. In one embodiment, effective doses of the hapten-carrier conjugate can range from about 1 μg to about 100 mg per dose, or from about 10 μg to about 50 mg per dose, or from about 50 μg to about 10 mg per dose or from about 0.5 mg to about 5 mg per dose (e.g., about 3 mg per dose).

Any effective regimen for administering the T_{H}1-biasing adjuvant, and the hapten-carrier conjugate can be used. For example, the T_{H}1-biasing adjuvant and the hapten-carrier conjugate can be administered as single doses, or they can be divided (i.e., fractionated) and administered as a multiple-dose daily regimen. Further, a staggered regimen, for example, one to five days per week can be used as an alternative to daily treatment.

In exemplary embodiments, the ligand-immunogen conjugate and therapeutic factor can be administered as single doses, or they can be divided and administered as a multiple-dose daily regimen. Further, a staggered regimen, for example, one to six days per week can be used as an alternative to daily treatment. In one embodiment of the invention the host is treated with multiple injections of the ligand-immunogen conjugate and the therapeutic factor to eliminate the population of pathogenic cells. In one embodiment, the host is injected multiple times (e.g., about 2 up to about 50 times) with the ligand-immunogen conjugate, for example, at 12-72 hour intervals or at 48-72 hour intervals. Additional injections of the ligand-immunogen conjugate can be administered to the patient at an interval of days or months after the initial injections(s) and the additional injections prevent recurrence of disease. Alternatively, the initial injection(s) of the ligand-immunogen conjugate may prevent recurrence of disease.

In one embodiment, a method is provided of treating a host animal to eliminate pathogenic cells. The method comprises the steps of administering to the host animal a hapten-carrier conjugate, administering to the host animal a T_{H}-1 biasing adjuvant wherein the ratio of the hapten-carrier conjugate to the T_{H}-1 biasing adjuvant on a weight to weight basis ranges from about 1:10 to about 1:1, and administering to the host animal a ligand conjugated to the hapten wherein the administration of the ligand-hapten conjugate is initiated during the first cycle of therapy with the hapten-carrier conjugate. Illustratively, this method can be used to reduce the probability of occurrence of adverse reactions (e.g., rashes, itching, flushing) that may indicate an allergic response. As used herein, “the first cycle of therapy” means the first, second, third, or fourth week of administration of the hapten-carrier conjugate whether or not the administration of the hapten-carrier conjugate is continuous during the first cycle of therapy.

Illustratively, in this embodiment, the pathogenic cells can be cancer cells or activated immune cells, such as macrophages or monocytes. In one embodiment, administration of the ligand-hapten conjugate is initiated during the first week of therapy with the hapten-carrier conjugate. In another embodiment, administration of the ligand-hapten conjugate is initiated during the second week of therapy with the hapten-carrier conjugate. In other embodiments, the ligand-hapten conjugate can be administered at the start of any week of administration of the hapten-carrier conjugate as long as the administration of the ligand-hapten conjugate is initiated before the first cycle of therapy with the hapten-carrier conjugate is complete. In various embodiments, other therapeutic factors, such as cytokines, can be administered along with the ligand-hapten conjugates. In another embodiment, the ligand-hapten conjugate dose (e.g., 0.3 mg/kg (qd×5)) can be fractionated and the ligand-hapten conjugate can be administered as fractionated doses on a daily basis (e.g., 60%, 30%, and 10% of the 0.3 mg/kg dose).

In various illustrative embodiments, the ratio of the hapten-carrier conjugate to the T_{H}-1 biasing adjuvant on a weight to weight basis ranges from about 1:8 to about 1:1, about 1:6 to about 1:1, about 1:4 to about 1:1, about 1:3 to about 1:1, or is about 1:3 or about 1:2.5 (e.g., 1.2 mg to 3 mg per day). In one embodiment, the hapten-carrier conjugate and the adjuvant can be mixed at a weight to weight ratio of about 1:3 or about 1:2.5 or about 1:2 within about 5 minutes to about 1 hour of administration to the patient to avoid micelle formation.

In one embodiment, the hapten-carrier conjugate has the formula

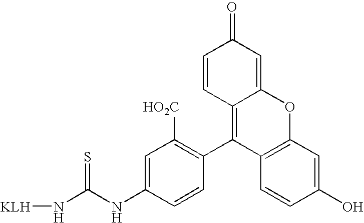

wherein KLH is keyhole limpet hemocyanin, and the ligand-hapten conjugate has the formula

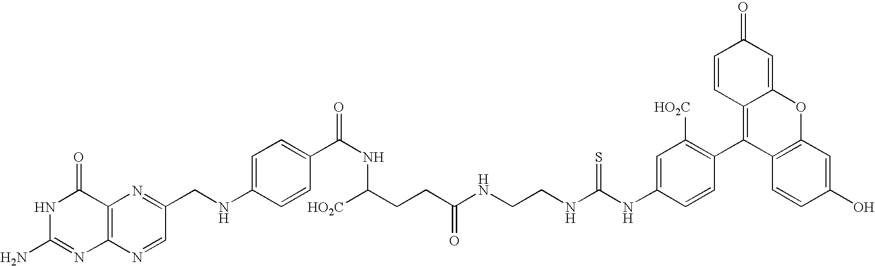

or pharmaceutically acceptable salts thereof.

In another embodiment, a method of treating a host animal to eliminate pathogenic cells is provided. The method comprises the steps of administering to the host animal a hapten-carrier conjugate, administering to the host animal a T_{H}-1 biasing adjuvant, and administering to the host animal a ligand conjugated to a hapten wherein the ligand-hapten conjugate is administered during the first cycle of therapy with the hapten-carrier conjugate. In one embodiment where the ligand is folate, or an analog or derivative of folate, a folate-targeted chelator radiolabeled with ^{99m}Te can be used to determine whether the patient has folate-receptor positive tumors (see U.S. Patent Application Publication No. 20040033195, incorporated herein by reference).

Illustratively, this method can be used to reduce the probability of occurrence of adverse reactions (e.g., rashes, itching, flushing) that may indicate an allergic response. In various aspects, the pathogenic cells can be cancer cells or activated immune cells, such as macrophages or monocytes.

In one embodiment, administration of the ligand-hapten conjugate is initiated during the first week of therapy with the hapten-carrier conjugate. In another embodiment, administration of the ligand-hapten conjugate is initiated during the second week of therapy with the hapten-carrier conjugate. In other embodiments, the ligand-hapten conjugate can be administered at the start of any week of administration of the hapten-carrier conjugate as long as the administration of the ligand-hapten conjugate is initiated before the first cycle of therapy with the hapten-carrier conjugate is complete. In various embodiments, other therapeutic factors, such as cytokines, can be administered along with the ligand-hapten conjugates. In another embodiment, the ligand-hapten conjugate dose (e.g., 0.3 mg/kg (qd×5)) can be fractionated and the ligand-hapten conjugate can be administered as fractionated doses on a daily basis (e.g., 60%, 30%, and 10% of the 0.3 mg/kg dose). In illustrative aspects, the hapten-carrier conjugate (in one aspect in combination with an adjuvant, such as GPI-0100), the ligand-hapten conjugate, and the therapeutic factor can be administered once weekly, TIW (three times a week), daily, or using any other useful dosing schedule.

In one embodiment of this method, the hapten-carrier conjugate and the adjuvant can be mixed within about 5 minutes to about 1 hour of administration to the patient to avoid micelle formation. In one embodiment, the hapten-carrier conjugate has the formula

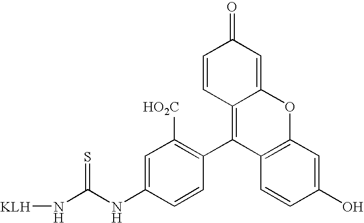

wherein KLH is keyhole limpet hemocyanin (conjugate referred to as KLH-FITC), and the ligand-hapten conjugate has the formula

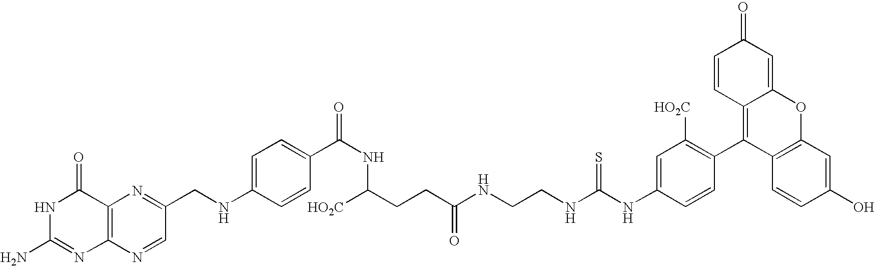

(conjugate referred to as folate-FITC) or pharmaceutically acceptable salts thereof.

In various embodiments, the therapeutic factor may be administered to the host animal prior to, after, or at the same time as the ligand-immunogen conjugate and the therapeutic factor may be administered as part of the same composition containing the conjugate or as part of a different composition than the ligand-immunogen conjugate. Any such therapeutic composition containing the therapeutic factor at a therapeutically effective dose can be used in the present invention. In one embodiment, more than one type of ligand-immunogen conjugate may be used. For example, the host animal may be preimmunized with both fluorescein isothiocyanate and dinitrophenyl and subsequently treated with fluorescein isothiocyanate and dinitrophenyl linked to the same or different ligands in a co-dosing protocol.

Illustratively, the ligand-immunogen (e.g., hapten) conjugate, the therapeutic factor, the adjuvant, and the hapten-carrier conjugate can be injected parenterally and such injections can be intraperitoneal injections, subcutaneous injections, intramuscular injections, intravenous injections or intrathecal injections. In another embodiment, the ligand-immunogen (e.g., hapten) conjugate, the therapeutic factor, the adjuvant, and the hapten-carrier conjugate can be delivered using a slow pump. Examples of parenteral dosage forms include aqueous solutions of the active agent in well-known pharmaceutically acceptable liquid carriers such as liquid alcohols, glycols (e.g., polyethylene glycols), glucose solutions (e.g., 5%), esters, amides, sterile water, buffered saline (including buffers like phosphate or acetate; e.g., isotonic saline). Additional exemplary components include vegetable oils, gelatin, lactose, amylose, magnesium stearate, talc, silicic acid, paraffin, and the like. In another aspect, the parenteral dosage form can be in the form of a reconstitutable lyophilizate comprising the dose of the ligand-immunogen (e.g., hapten) conjugate, the therapeutic factor, the adjuvant, or the hapten-carrier conjugate. In various aspects, solubilizing agents, local anaesthetics (e.g., lidocaine), excipients, preservatives, stabilizers, wetting agents, emulsifiers, salts, and lubricants can be used. In one aspect, any of a number of prolonged release dosage forms known in the art can be administered such as, for example, the biodegradable carbohydrate matrices described in U.S. Pat. Nos. 4,713,249; 5,266,333; and 5,417,982, the disclosures of which are incorporated herein by reference.

### Example 1

### Temperature Analysis in Balb/C Mice

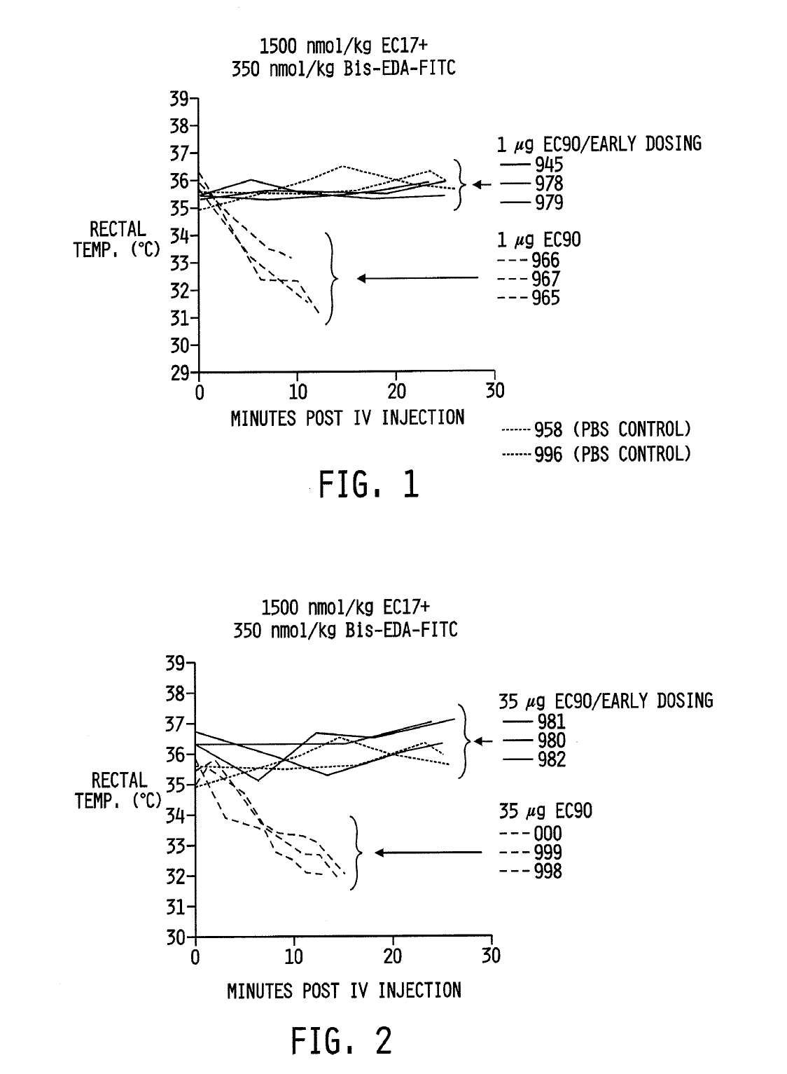

Female Balb/c mice were immunized 3 times at 1-week intervals against either 1 μg (FIG. 1) or 35 μg (FIG. 2) of EC90 (KLH-FITC; see FIG. 5) formulated with 100 μg GPI-0100. Bisfluorescein, was added to the EC17 (folate-FITC; see FIG. 4) composition (1500 nmol/kg EC17 plus 350 nmol/kg bisfluorescein). Bisfluorescein was added to enhance the allergenicity of the composition. The mice were intravenously challenged with 1500 nmol/kg EC17 plus 350 nmol/kg bisfluorescein. The mice were then monitored for any change in body temperature via a rectal probe to detect any apparent allergenicity.

Preparation of Injectates: The EC90 (KLH-FITC)/GPI-0100 solutions were made fresh prior to each vaccination to avoid micelle formation upon storage. The 1 μg EC90/GPI-0100 injectate (FIG. 1) was prepared by mixing 0.01 mg/ml EC90 and 1 mg/ml GPI-0100 in PBS, at pH 7.4 (0.1 ml per dose provided 1 μg KLH-FITC and 100 μg GPI-0100). The 35 μg EC90/GPI-0100 injectate (FIG. 2) was prepared by mixing 0.35 mg/ml EC90 and 1 mg/ml GPI-0100 in PBS, at pH 7.4 (0.1 ml per dose provided 35 μg KLH-FITC and 100 μg GPI-0100). The bisfluorescein-spiked EC17 injectate was prepared by mixing 0.244 ml of the EC17 stock solution with 2.331 ml of the bisfluorescein stock solution, and 2.425 ml PBS, at pH 7.4, for each 5 ml volume. For IV or SC administration, 0.1 ml per ˜20 g mouse provided 1500 nmol/kg EC17 plus 350 nmol/kg bisfluorescein.
Vaccination: Mice were immunized subcutaneously at adjacent sites (50 μl/site) at the base of the tail with 100 μl of the 1 μg or 35 μg EC90/GPI-0100 injectate. Seven and fourteen days later, the mice were given two booster doses injected on their back or the back of the neck.
Early Dosing with Bisfluorescein-Spiked EC17 Injectate: Mice were treated with 1500 nmol/kg EC17 plus 350 nmol/kg bisfluorescein on days 7 to 11, days 14 to 18, and day 21.
Late Dosing with Bisfluorescein-Spiked EC17 Injectate: On about day 22, mice were intravenously challenged with PBS or 1500 nmol/kg EC17 plus 350 nmol/kg bisfluorescein. The body temperature of each mouse was measured using a rectal probe designed specifically for mice (RET-3, Thermocouple Thermometer). The baseline temperature was taken before each animal was warmed up for IV injection, immediately prior to injection, and for approximately 30 minutes post challenge (as frequently as necessary).
Results: EC17 (1500 nmol/kg) spiked with bisfluorescein (350 nmol/kg) caused a decrease in temperature in mice immunized against the two EC90 doses, except where early dosing with EC17+bisfluorescein had been performed. By dosing mice early with a bisfluorescein-contaminated EC17, responses indicating apparent allergic reactions to the spiked bisfluorescein were prevented. Also, EC90 alone (in the absence of a challenge with EC17+bisfluorescein; i.e., only EC17 was added and EC17 was added in a late dosing protocol) caused a decrease in temperature in mice when administered at 1 μg (resulting in a ratio of EC90 to GPI-0100 on a weight to weight basis of about 1:100), but not at 35 μg (ratio of EC90 to GPI-0100 on a weight to weight basis of about 1:2.5.

### Example 2

### Effect of Ligand Conjugates on Tumor Volume for Mice with Breast Tumor Implants

Two regimens were tested. In the first regimen, six to eight-week old (˜20-22 grams) female Balb/c mice were immunized with fluorescein isothiocyanate (FITC)-labeled keyhole limpet hemocyanin (KLH; see FIG. 5) at 35 μg/dose using a saponin adjuvant (e.g., GPI-0100; 100 μg/dose) at days 1, 15, and 29. On day 23, each animal was injected with 2.5×10⁵ 4T1c2 cells (a breast tumor cell line). Cancer loci were then allowed to grow. From days 42-60, all animals were injected daily ((qd×5)₃; days 42-46, 49-53, and 56-60) with either phosphate buffered saline (PBS) or 500 nmol/kg of FITC-conjugated to folic acid via a gamma carboxyl-linked ethylene diamine bridge (see FIG. 4). The animals were injected on the same days with 20,000 U/dose of recombinant human IL-2. The animals were injected (TIW)₃ with IL-2 in the same weeks as the animals were injected with folate-FITC.

In the second regimen, six to eight-week old (˜20-22 grams) female Balb/c mice were immunized with fluorescein isothiocyanate (FITC)-labeled keyhole limpet hemocyanin (KLH) at 35 μg/dose using a saponin adjuvant (e.g., GPI-0100; 100 μg/dose) at days 1, 15, and 29. On day 5, each animal was injected with 2.5×10⁵ 4T1c2 cells. Cancer loci were then allowed to grow. From days 8-50, all animals were injected daily ((qd×5)₆) with either phosphate buffered saline (PBS) or 500 nmol/kg of FITC-conjugated to folic acid via a gamma carboxyl-linked ethylene diamine bridge. The animals were injected daily on days 32-50 with 20,000 U/dose of recombinant human IL-2. The animals were injected (TIW)₃ with IL-2 in the same weeks as the animals were injected with folate-FITC.

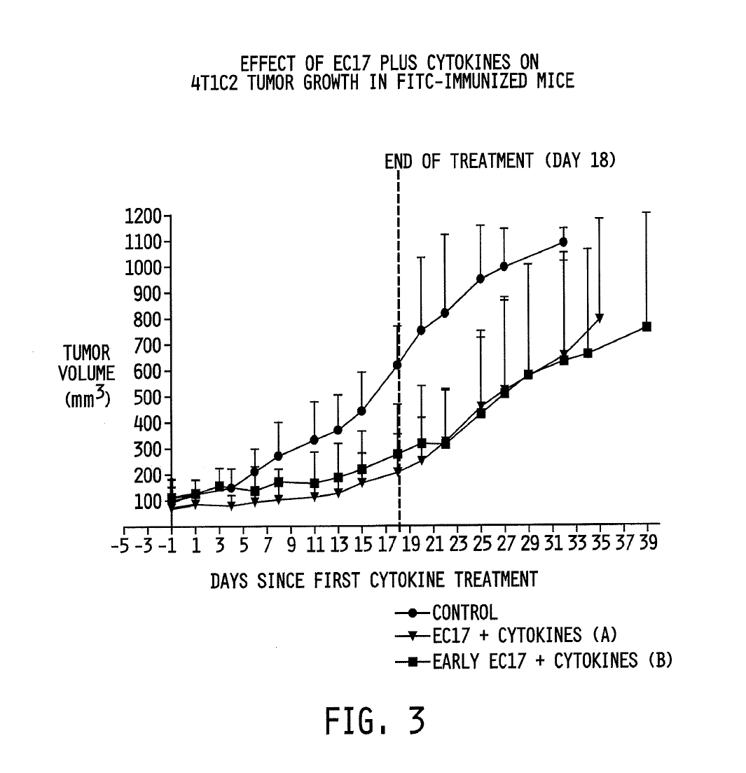

The efficacy of this immunotherapy was then evaluated by monitoring tumor volume as a function of time for folate-FITC treated mice compared to control animals. As shown in FIG. 3, tumor volume for mice was decreased with the immunotherapy and tumor volume was similar regardless of the dosing protocol (early or late) used to administer folate-FITC. Accordingly, the “early dosing protocol” with folate-FITC was effective in decreasing tumor volume.

### Example 3

### Synthesis of KLH-FITC and Folate-FITC

Folate-FITC was synthesized and purified as described in Kennedy, et al. in Pharmaceutical Research, Vol. 20(5), 2003 and in WO2006/101845, each incorporated herein by reference. EC17 was stored as a frozen solution of 5.5 mg/ml in PBS, pH 7.4. EC90 (KLH-FITC) solid (83% protein content) had a labeling ratio of ˜129 μmol FITC per gram of KLH. The stock solution was made in PBS, pH 7.4 at 2.5 mg/ml and sterile filtered with a 0.22 μm syringe filter. KLH-FITC was synthesized using methods similar to those for folate-FITC.

### Example 4

### Dosing Protocol

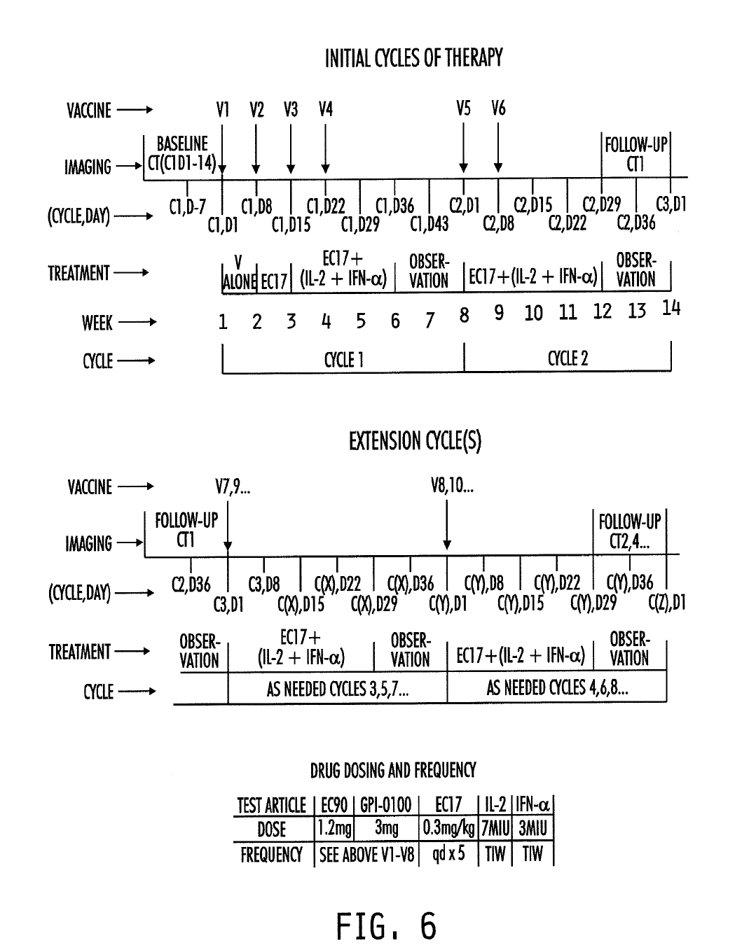

FIG. 6 shows an exemplary “early dosing protocol” used in humans for the method described herein to reduce the probability of adverse reactions (e.g., rashes, flushing, itching) that indicate an allergy. V1 through V10 indicate injections with EC90 (KLH-FITC). The weeks for the therapeutic cycles are shown and the days of the weeks during the cycles are shown as D1, D8, D15, etc. The cycles are shown as C1, C2, C3, etc. The weeks, cycles, and days on which EC90 (V1, V2, etc.), EC17 (folate-FITC), and EC17+cytokines were administered are shown. A table showing the drug dose and frequency of dosing is also included in FIG. 6. EC90, GPI-0100, EC17, IL-2, and IFN-α were dosed at 1.2 mg, 3 mg, 0.3 mg/kg, 7 MIU, and 3 MIU, respectively.

### Example 5

### Dosing Protocol

Another exemplary “early dosing protocol” includes the following steps. A folate-targeted chelator (0.1 mg administered IV (in the vein)) radiolabeled with ^{99m}Te is used to determine whether the patient has folate-receptor positive tumors (see U.S. Patent Application Publication No. 20040033195, incorporated herein by reference). KLH-FITC (1.2 mg in combination with adjuvant GPI-0100) is administered subcutaneously weekly (i.e., once per week) for 4 consecutive weeks during the first cycle of treatment, weekly for 2 consecutive weeks during the second cycle and once for each additional cycle. GPI-0100 adjuvant is administered in combination with KLH-FITC (GPI-0100 is at 3.0 mg) subcutaneously weekly for 4 consecutive weeks during the first cycle of treatment, weekly for 2 consecutive weeks during the second cycle and once for each additional cycle. Folate-FITC (0.3 mg/kg) is administered subcutaneously 5 days per week (Monday through Friday) for 4 consecutive weeks for the first two treatment cycles and then 3 days per week (Monday, Wednesday, and Friday) for 3 consecutive weeks for each additional cycle. IL-2 (7.0 MIU) is administered subcutaneously 3 times per week (Monday, Wednesday, and Friday) for 4 consecutive weeks during the first 2 cycles of treatment, then 2.5 MIU of IL-2 is administered subcutaneously 3 times per week (Monday, Wednesday, and Friday) for 3 consecutive weeks for each additional cycle. IFN-α (3.0 MIU) is administered subcutaneously 3 times per week (Monday, Wednesday, and Friday) for 4 consecutive weeks during the first 2 cycles of treatment, then 3.0 MIU of IFN-α is administered subcutaneously 3 times per week (Monday, Wednesday, and Friday) for 3 consecutive weeks for each additional cycle.

### Example 6

### Active Systemic Anaphylaxis Assay in Mice Immunized Against EC90 Formulated with GPI-0100

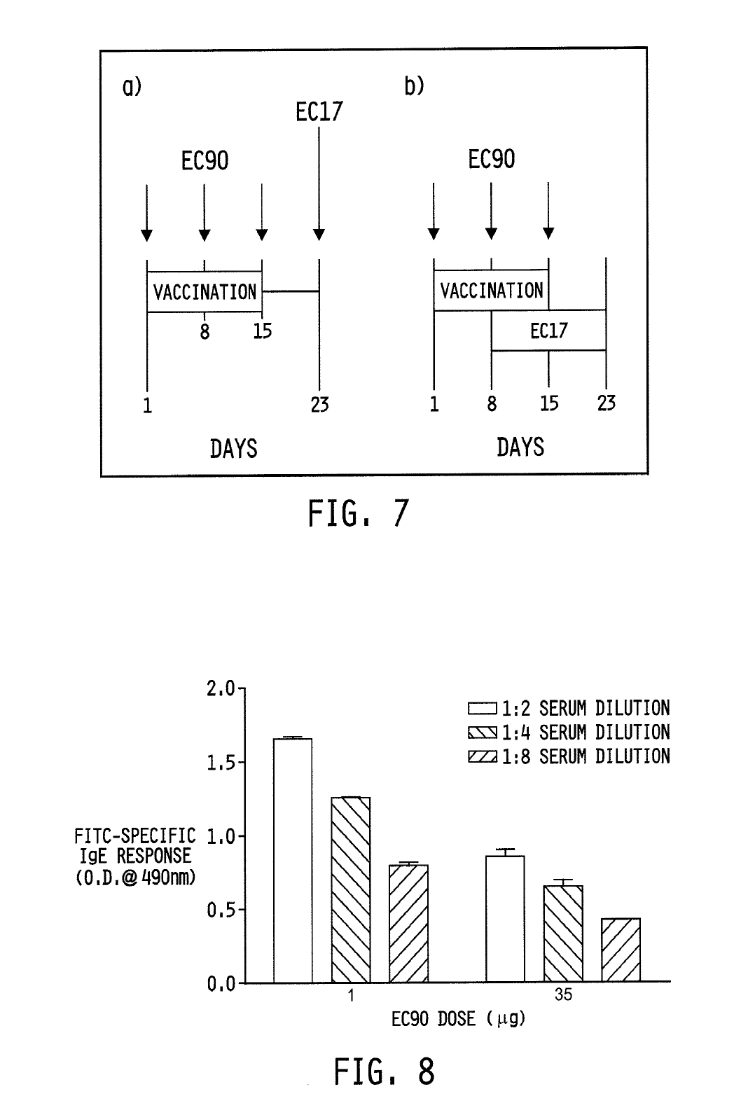

Female Balb/c mice were immunized three times, on Days 1, 8, and 15. A single dose of EC17 was intravenously administered on Day 23 (FIG. 7, Panel a). Mice were de-sensitized with multiple subcutaneous doses of EC17 on Days 8-12, 15-19, and 22 (FIG. 7, Panel b). On Day 23, the mice were intravenously challenged with EC 17 as usual. Following EC 17 challenge, the body temperature was measured using a rectal probe (RET-3, Thermocouple Thermometer). The baseline temperature was taken before each animal was warmed up for intravenous injection, immediately prior to injection, and for ˜30 min post challenge (as frequent as necessary). Animals were euthanized by CO₂ when they displayed signs of shock with no activity after prodding (usually their body temperature had drooped by −3° C. or below).

### Example 7

### Anti-FITC IGE Antibody Production in FITC-Immunized Mice

Female Balb/c mice (n=3) were immunized against various doses of EC90 plus 100 μg GPI-0100 on Days 1, 8, and 15. The serum was pooled at equal volumes from individual animals in each group on Day 29. The relative levels of anti-FITC IgE antibody were compared using a capture ELISA assay (FIG. 8). Briefly, 96-well plates were coated with a rat anti-mouse IgE capture mAb. After blocking non-specific binding, the plates were incubated with FITC-antiserum followed by biotinylated BSA-FITC and streptavidin-horseradish peroxidase.

### Example 8

### Active Systemic Anaphylaxis Assay in Guinea Pigs Immunized Against EC90 Plus GPI-0100 Adjuvant

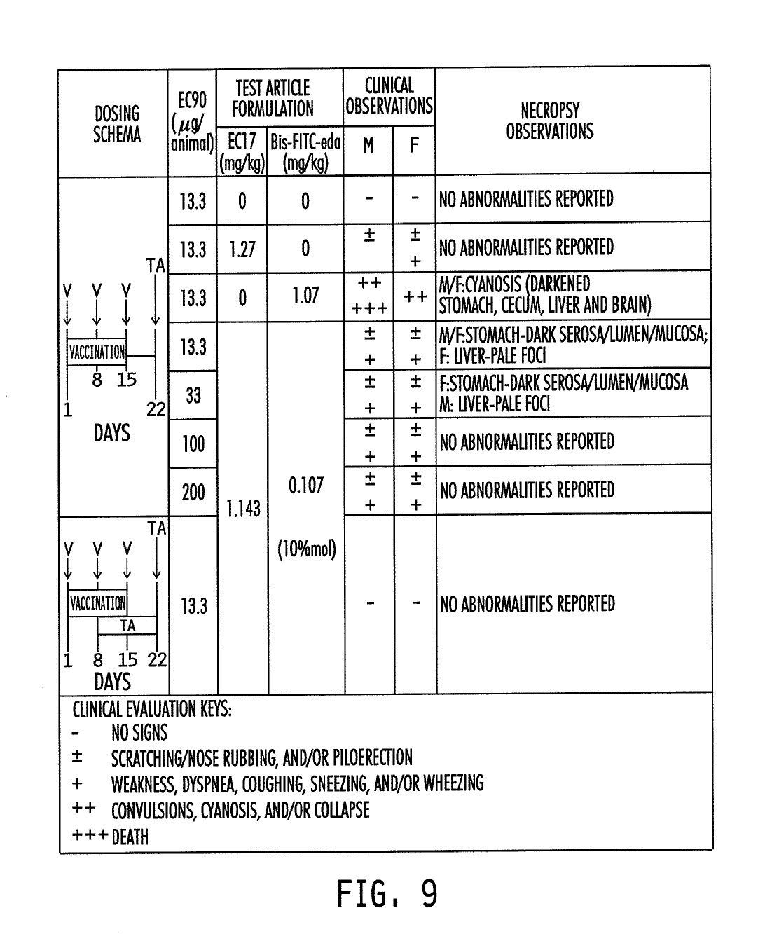

Male and female guinea pigs (1 per sex per group) were immunized three times, on Days 1, 8, and 15, with various doses of EC90 plus 0.5 mg GPI-0100. A single dose of test article (EC17+/− Bis-FITC-eda) was administered (s.c.) on Day 22. Guinea pigs were de-sensitized with multiple doses of EC 17 spiked with 10% (mole) Bis-FITC-eda on Days 8-12, and 15-19. On Day 22, these animals were s.c. challenged with the same EC17/Bis-FITC-eda formulation. Clinical observations were generally taken for 1.5-2 hours post challenge. Animals were euthanized when they displayed signs of anaphylactic shock. Complete macroscopic postmortem examinations were performed on all animals (FIG. 9). The results show that early dosing with EC 17 and increasing the dose of KLH-FITC reduces allergenicity in animals.

1. A method of treating a host animal to eliminate pathogenic cells, the method comprising the steps of,
administering to the host animal a hapten-carrier conjugate,
administering to the host animal a T_{H}-1 biasing adjuvant wherein the ratio of the hapten-carrier conjugate to the T_{H}-1 biasing adjuvant on a weight to weight basis ranges from about 1:10 to about 1:1; and
administering to said host animal a ligand conjugated to the hapten wherein administration of the ligand-hapten conjugate is initiated during the first cycle of therapy with the hapten-carrier conjugate.

2. The method of claim 1 wherein the pathogenic cells are cancer cells.

3.-4. (canceled)

5. The method of claim 1 wherein administration of the ligand-hapten conjugate is initiated during the first or second week of therapy with the hapten-carrier conjugate or at a later time wherein the later time is before the first cycle of therapy with the hapten-carrier conjugate is complete.

6. (canceled)

7. The method of claim 1 wherein the ligand is selected from the group consisting of folic acid and other folate receptor-binding ligands.

8.-10. (canceled)

11. The method of claim 1 wherein the hapten is an organic molecule having a molecular weight less than 20,000 daltons.

12. The method of claim 11 wherein the organic molecule is selected from the group consisting of fluorescein, a nitrophenyl, and a polynitrophenyl.

13. (canceled)

14. The method of claim 1 further comprising the step of administering an immune stimulant to the host animal.

15.-16. (canceled)

17. The method of claim 14 wherein the immune stimulant is a cytokine comprising IL-2, IL-12, IL-15, or combinations thereof, in combination with IFN-γ or IFN-α.

18.-27. (canceled)

28. The method of claim 1, wherein the hapten-carrier conjugate has the formula: 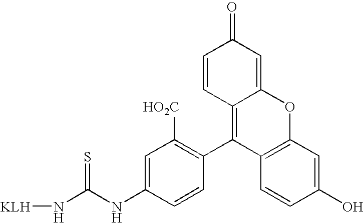
wherein KLH is keyhole limpet hemocyanin, and the ligand-hapten conjugate has the formula: 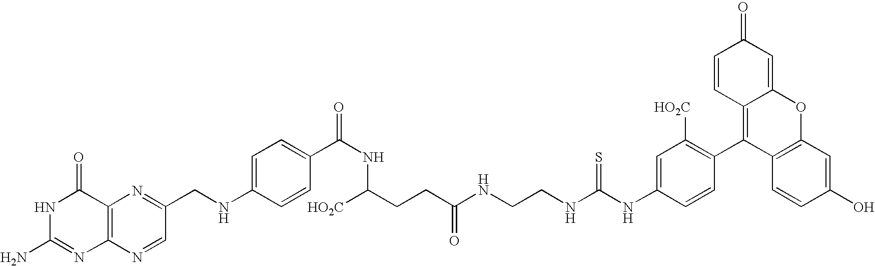
or pharmaceutically acceptable salts thereof.

29. A method of treating a host animal to eliminate pathogenic cells, the method comprising the steps of,
administering to the host animal a hapten-carrier conjugate,
administering to the host animal a T_{H}-1 biasing adjuvant; and
administering to said host animal a ligand conjugated to a hapten wherein the administration of the ligand-hapten conjugate is initiated during the first cycle of therapy with the hapten-carrier conjugate.

30. The method of claim 29 wherein the pathogenic cells are cancer cells.

31.-32. (canceled)

33. The method of claim 29 wherein administration of the ligand-hapten conjugate is initiated during the first week of therapy with the hapten-carrier conjugate or at a later time wherein the later time is before the first cycle of therapy with the hapten-carrier conjugate is complete.

34. (canceled)

35. The method of claim 29 wherein the ligand is selected from the group consisting of folic acid and other folate receptor-binding ligands.

36.-38. (canceled)

39. The method of claim 29 wherein the hapten is an organic molecule having a molecular weight less than 20,000 daltons.

40. The method of claim 39 wherein the organic molecule is fluorescein, a nitrophenyl, or a polynitrophenyl.

41. (canceled)

42. The method of claim 29 further comprising the step of administering an immune stimulant to the host animal.

43.-44. (canceled)

45. The method of claim 42 wherein the immune stimulant is a cytokine comprising IL-2, IL-12, IL-15, or combinations thereof, in combination with IFN-γ or IFN-α.

46.-47. (canceled)

48. The method of claim 29 wherein the adjuvant is a quillajasaponin adjuvant.

49.-50. (canceled)

51. The method of claim 29, wherein the hapten-carrier conjugate has the formula: 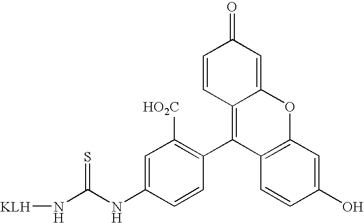
wherein KLH is keyhole limpet hemocyanin, and the ligand-hapten conjugate has the formula: 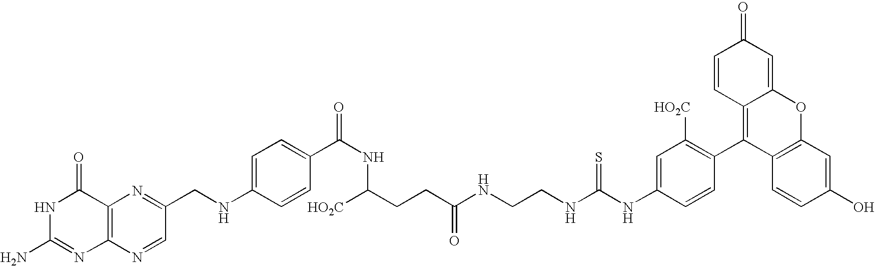
or pharmaceutically acceptable salts thereof.
